(19) United States
(12) Patent Application Publication
(10) Pub. No.: US 2024/0269160 A1
(43) Pub. Date: Aug. 15, 2024

(54) Use Of Citicoline In Regulating Respiratory Chain Function Related Protein Level And Preparing Drug For Preventing And Treating Respiratory Chain Dysfunction

(71) Applicant: Tongji Hospital, Tongji Medical College, Huazhong University of Science and Technology, Wuhan City (CN)

(72) Inventors: Chen CHEN, Wuhan City (CN); Huaping LI, Wuhan City (CN); Daowen WANG, Wuhan CIty (CN); Beibei DAI, Wuhan City (CN)

(21) Appl. No.: 18/418,193

(22) Filed: Jan. 19, 2024

(30) Foreign Application Priority Data
Feb. 2, 2023 (CN) 202310099279.2

(51) Int. Cl. A61K 31/7068; A01K 67/027; A61P 43/00; C12N 15/113; C12N 15/86

(52) U.S. Cl. CPC A61K 31/7068; A01K 67/027; A61P 43/00; C12N 15/113; C12N 15/86; A01K 2207/05; A01K 2267/0306; C12N 2310/122; C12N 2750/14143

(57) ABSTRACT

The present invention of use of citicoline in regulating respiratory chain function related protein level and preparing drug for preventing and treating respiratory chain dysfunction belongs to the field of drug. The present invention provides use of citicoline in regulating respiratory chain function related protein level, and also provides use of citicoline in preparing drug for preventing and treating respiratory chain dysfunction. The present invention also provides a shRNA causing respiratory chain dysfunction, its reverse complementary sequence is shown as SEQ ID NO. 2. The present invention also provides use of shRNA in preparing a respiratory chain dysfunction animal model, as well as its preparation method. It's confirmed by experiments that citicoline can effectively reverse molecular level symptoms of respiratory chain dysfunction and can be used as a drug for treating respiratory chain dysfunction.

### CROSS-REFERENCE TO RELATED APPLICATION

This application claims priority to and the benefit of Chinese patent Application No. 202310099279.2 filed Feb. 2, 2023. The entire contents of Chinese patent Application No. 202310099279.2 and the English translation of Chinese patent Application No. 202310099279.2 are incorporated herein by reference.

### SEQUENCE LISTING

This application contains references to amino acid sequences and/or nucleic acid sequences which have been submitted concurrently herewith as the sequence listing .xml file entitled “000006us_SequenceListing.xml”, file size 3,254 bytes, created on Jan. 17, 2024. The aforementioned sequence listing is hereby incorporated by reference in its entirety pursuant to 37 C.F.R. § 1.52(e)(5).

### TECHNICAL FIELD

The present invention relates to a field of drug, in particular to use of citicoline in regulating respiratory chain function related protein level and preparing drug for preventing and treating respiratory chain dysfunction.

### BACKGROUND

A respiratory chain is a continuous reaction system composed of a series of hydrogen transfer reactions and electron transfer reactions arranged in a certain order. It hands off paired hydrogen atoms from metabolites to oxygen to produce water, while ATP is produced.

NDUFA8, UQCRC2, and COXIV are known respiratory chain function related proteins.

1,2-diacyl-sn-glycero-3-phosphocholine (PC) is an amphipathic molecule composed of a hydrophilic head and a hydrophobic tail. It is a type of phospholipid with a choline group inserted at the head. The 1,2-diacyl-sn-glycero-3-phosphocholine is an important component of biofilms.

Phosphatidylethanolamine (PE), also known as brain phospholipids, is one of the important phospholipids that form biofilms. It mainly exists in the brain, nerves, microorganisms, and soybeans, playing important roles in signal transduction and maintaining life functions.

PC and PE also play important roles in respiratory chain function. Respiratory chain dysfunction is mainly manifested as a decrease in content of PC, PE, and ATP in body at the molecular level. Respiratory chain dysfunction can lead to various diseases.

Therefore, it is necessary to develop new drugs for preventing and treating respiratory chain dysfunction in this field.

Citicoline is a nucleoside derivative. The reported therapeutic uses of Citicoline include improving consciousness state and electroencephalogram of consciousness disorders after head trauma or brain surgery, promoting recovery of upper limb motor function in stroke hemiplegic patients, and having a certain effect on promoting brain function recovery and awakening.

However, there have been no reports in this field on use of citicoline in regulating respiratory chain function related protein level, preventing and treating respiratory chain dysfunction.

### SUMMARY

Based on above requirements and research gaps in this field, the present invention provides use of citicoline in regulating respiratory chain function related protein level and preparing drug for preventing and treating respiratory chain dysfunction.

The technical solution of the present invention is as follows:

Use of citicoline in regulating respiratory chain function related protein level.
said respiratory chain function related protein is selected from groups consisting of NDUFA8, UQCRC2, COXIV;
said regulating refers to increasing levels of NDUFA8 and/or UQCRC2 and/or COXIV in respiratory chain dysfunction animal model;
preferably, the respiratory chain dysfunction animal model is obtained by overexpressing shRNA in animal body; a reverse complementary sequence of the shRNA is shown as SEQ ID NO. 2;
preferably, DNA sequence corresponding to the shRNA is shown as SEQ ID NO. 1;
preferably, shRNA induces respiratory chain dysfunction in animal by downregulating levels of NDUFA8 and/or UQCRC2 and/or COXIV;
preferably, the respiratory chain dysfunction refers to a decrease in content of PC and/or PE and/or ATP in the animal body;
preferably, the animal is selected from: mouse, rabbit, monkey.

Use of citicoline in preparing drug for preventing and treating respiratory chain dysfunction.

The drug comprises: pharmacodynamically active ingredients; The pharmacodynamically active ingredients include: citicoline;
preferably, the drug further comprises: pharmaceutically acceptable excipients;
preferably, said preventing and treating refer to: citicoline relieves and/or improves symptoms of respiratory chain dysfunction;
preferably, the symptoms are selected from: levels of PC and/or PE and/or ATP decreased, and/or, levels of NDUFA8 and/or UQCRC2 and/or COXIV decreased.

A shRNA causing respiratory chain dysfunction, its reverse complementary sequence is shown as SEQ ID NO. 2.

DNA sequence corresponding to the shRNA is shown as SEQ ID NO. 1;
preferably, said causing respiratory chain dysfunction refers to levels of NDUFA8 and/or UQCRC2 and/or COXIV protein are downregulated by overexpressing shRNA in animal body to lead respiratory chain dysfunction in animal;
preferably, the respiratory chain dysfunction refers to levels of PC and/or PE and/or ATP in the animal body decreased.

Use of shRNA in preparing a respiratory chain dysfunction animal model, a reverse complementary sequence of shRNA is shown as SEQ ID NO. 2.

DNA sequence corresponding to the shRNA is shown as SEQ ID NO. 1;
preferably, shRNA induces respiratory chain dysfunction in animal by downregulating levels of NDUFA8 and/or UQCRC2 and/or COXIV;
preferably, the respiratory chain dysfunction refers to a content of PC and/or PE and/or ATP in animal body decrease;
preferably, the animal is selected from: mouse, rabbit, monkey.

A method for preparing a respiratory chain dysfunction animal model, comprising the following step: downregulating levels of NDUFA8 and/or UQCRC2 and/or COXIV in animal by shRNA whose reverse complementary sequences is shown as SEQ ID NO. 2.

The respiratory chain dysfunction refers to content of PC and/or PE and/or ATP in animal body decreased;
preferably, connecting DNA sequence corresponding to shRNA which is shown as SEQ ID NO. 1 to expression vector to obtain a recombinant expression vector;
preferably, transforming the recombinant expression vector into the host to obtain a transformant;
preferably, transferring the transformant into the animal;
preferably, the expression vector is selected from pAAV-TNT vector;
preferably, the host is selected from a virus or cell;
preferably, the virus is selected from an adenovirus;
preferably, the cell is selected from DH5a competent cell or 293T cell;
preferably, the animal is selected from: mouse, rabbit, monkey.

On the one hand, the present invention provides a use of citicoline in regulating respiratory chain function related protein level.

On the other hand, the present invention provides a use of citicoline in preparing drug for preventing and treating respiratory chain dysfunction.

In some countries or regions where patent laws permit, the present invention also claims use of citicoline for preventing and treating respiratory chain dysfunction.

Optionally, the respiratory chain dysfunction is caused by decreased expression of NDUFA8, UQCRC2, and COXIV.

Optionally, the drug is a drug that improves respiratory chain dysfunction.

Improving metabolism can be reflected in an increase in content of phosphatidylcholine (PC) and phosphatidylethanolamine (PE).

Improving respiratory chain function can be reflected in an increase in ATP content.

Optionally, the drug may be capsule, tablet, granule, pill, or oral liquid.

Optionally, the drug also includes pharmaceutically acceptable excipients or additives.

Above drug can significantly increase the expression of NDUFA8 protein, UQCRC2 protein, and COXIV protein.

The present invention also provides a method for treating and/or preventing and/or alleviating and/or improving respiratory chain dysfunction, including administering citicoline to recipient animals for treatment and/or prevention and/or alleviation and/or improvement of respiratory chain dysfunction.

Optionally, the respiratory chain dysfunction is caused by decreased expression of NDUFA8, UQCRC2, and COXIV.

A respiratory chain dysfunction animal model caused by decreased expression of NDUFA8, UQCRC2, and COXIV mentioned above can be a shRNA intervention mice, whose levels of NDUFA8, UQCRC2, and COXIV proteins significantly decrease. For example, a recombinant adeno-associated virus (rAAV) vector carrying shRNA is introduced into mice through tail vein injection.

The shRNA refers to a hairpin structure nucleic acid that reduces mRNA content or reduces protein levels through RNA interference technology.

Optionally, the shRNA intervention in mice includes using an rAAV vector carrying shRNA to reduce expression of NDUFA8, UQCRC2, and COXIV proteins in the recipient mice.

The present invention proposes for the first time use of citicoline in treating respiratory chain dysfunction, which can improve respiratory chain dysfunction. The respiratory chain dysfunction is caused by downregulation of NDUFA8 and/or UQCRC2 and/or COXIV levels in animal by shRNA with reverse complementary sequences as shown in SEQ ID NO. 2, specifically manifested as a decrease in PC, PE, and ATP levels in the animal body. Through experiments, it was found that after treatment with citicoline, levels of PC, PE, and ATP in respiratory chain dysfunction animal were increased, while the decreased levels of NDUFA8, UQCRC2, and COXIV proteins were restored. It's demonstrated that, citicoline can reverse molecular level symptoms of respiratory chain dysfunction and can be used as a drug for treating respiratory chain dysfunction.

### DESCRIPTION OF DRAWINGS

FIG. 1 shows a plasmid composition of pAAV-TNT-shRNA.

FIG. 2 shows an expression detection of NDUFA8 protein, UQCRC2 protein, and COXIV protein in experimental example 2.

FIG. 3 shows a detection of PC and PE content in experimental example 2.

FIG. 4 shows an ATP content detection in experiment example 2.

FIG. 5 shows an expression detection of NDUFA8 protein, UQCRC2 protein, and COXIV protein in experimental example 3.

FIG. 6 shows a detection of PC and PE content in experimental example 3.

FIG. 7 shows an ATP content detection in experimental example 3.

Marks in the figures are listed as follows:

rAAV-TNT shCON: mice injected with the control vector

rAAV-TNT-control; The control vector rAAV-TNT-control is the commercially available empty vector (pAAV-TNT vector).

rAAV-TNT-shRNA: mice injected with rAAV vector rAAV-TNT-shRNA carrying shRNA;

rAAV-TnT-shCON+control: blank control group mice injected with control vector rAAV-TNT-control at 8 weeks of age, and fed with physiological saline at 12 weeks of age;

rAAV-TnT-shRNA+control: positive control group mice injected with rAAV vector rAAV-TNT-shRNA carrying shRNA at 8 weeks of age, and fed with physiological saline at 12 weeks of age;

rAAV-TnT-shCON+CDP-choline: control group mice injected with the control vector rAAV-TNT-control at 8 weeks of age, and then fed with cytosine diphosphate choline at 12 weeks of age;

rAAV-TnT-shRNA+CDP-choline: experimental group mice injected with rAAV vector rAAV-TNT-shRNA carrying shRNA at 8 weeks of age, and then fed with choline diphosphate at 12 weeks of age.

### EMBODIMENTS

The technical solution and technical effects of the present invention will be described clearly and completely in the following content by combining with specific examples and experimental examples. Obviously, the described experimental examples and examples are only a part of the present invention's experimental examples and examples, not all of them. Based on the experimental examples and examples of the present invention, all other examples obtained by a person skilled in the art without creative work fall within protection scope of the present invention.

The experimental methods in the following embodiments and experimental examples, unless otherwise specified, are conventional methods and are carried out according to the techniques or conditions described in literatures in this field or in accordance with product manuals. The materials, reagents, etc. used in the following experimental examples can be commercially available unless otherwise specified.

Structural formula of citicoline in the following experimental example is formula 1, with CAS number 987-78-0. It is a product of MedChemExpress company (product number HY-B0739).

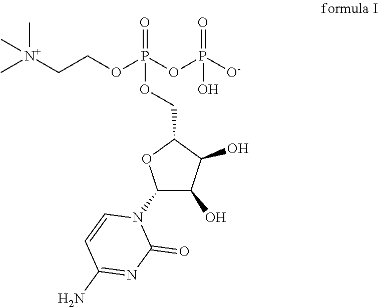 [SMILES: C[N+](C)(C)CCOP(=O)(OC[C@H]1O[C@@H](n2ccc(N)nc2=O)[C@H](O)[C@@H]1O)OP(=O)([O-])O]

Data was processed using GraphPad Prism 8 statistical software, and the experimental results were presented as mean±standard error using a two tailed t-test.

### Instrument and Equipment

Nanodrop® ND-1000 nucleic acid analyzer

ABI 9700 PCR instrument

ABI 7900HT fluorescence real-time quantitative PCR instrument

Beckman X-15R Low Temperature High Speed Centrifuge

### Reagents and Consumables

TRIZOL was purchased from Invitrogen;

The reverse transcription kit was purchased from Thermofish Company; SYBR Green was purchased from Thermofish Company;

EasyPure Plasmid MiniPrep Kit plasmid extraction kit was purchased from Beijing TransGen Biotech Company;

### Sources of Biomaterials

PAAV-TNT vector, DH5a competent cell and 293T cell (human embryonic renal epithelial cell) used in experiment example 1 can be commercially obtained;

C57BL/6 mice used in experiment example 2 and experiment example 3 were from Jiangsu GemPharmatech biotechnology Company Limited;

### Examples Group 1: Citicoline Regulating Protein Level

This group of examples provides use of citicoline in regulating respiratory chain function related protein level.

In specific examples, said respiratory chain function related protein is selected from groups consisting of NDUFA8, UQCRC2. COXIV.

In some embodiments, said regulating refers to increasing levels of NDUFA8 and/or UQCRC2 and/or COXIV in respiratory chain dysfunction animal model;
preferably, the respiratory chain dysfunction animal model is obtained by overexpressing shRNA in animal body; a reverse complementary sequence of the shRNA is shown as SEQ ID NO. 2;
preferably, DNA sequence corresponding to the shRNA is shown as SEQ ID NO. 1;
preferably, shRNA induces respiratory chain dysfunction in animal by downregulating levels of NDUFA8 and/or UQCRC2 and/or COXIV;
preferably, the respiratory chain dysfunction refers to a decrease in content of PC and/or PE and/or ATP in the animal body;
preferably, the animal is selected from: mouse, rabbit, monkey.

A person skilled in the art, teached by the present invention, can use citicoline to regulate levels of respiratory chain function related proteins other than NDUFA8, UQCRC2, and COXIV. Any use of citicoline for upregulating, downregulating, positive regulating, negative regulating, reducing, increasing, enhancing other respiratory chain function related protein level and/or knocking down, knocking out, silencing, or overexpressing genes of other respiratory chain function related protein falls within protection scope of the present invention.

### Examples Group 2: Citicoline Preventing and Treating Respiratory Chain Dysfunction

This group of example provides a use of citicoline in preparing drug for preventing and treating respiratory chain dysfunction.

In specific embodiments, the drug comprises: pharmacodynamically active ingredients; the pharmacodynamically active ingredients include: citicoline;

Preferably, the drug further comprises: pharmaceutically acceptable excipients;

In specific embodiments, the pharmaceutically acceptable excipients are selected from: solvents, projectiles, solubilizers, cosolvents, emulsifiers, colorants, adhesives, disintegrants, fillers, lubricants, wetting agents, osmotic pressure regulators, stabilizers, flow aids, flavor correction agents, preservatives, suspension aids, coating materials, aromatics, anti adhesives, integrators, penetration enhancers, pH regulators, buffering agents, plasticizers, surfactants, foaming agent, defoaming agent, thickener, packaging agent, moisturizing agent, absorbent, diluent, flocculant, anti flocculant, filter aid, release blocker.

In specific embodiments, said preparing is selected from groups consisting of synthetizing, amplifying, expressing, cloning, secreting, enriching, expanding, and reaction generating;
preferably, said preventing and treating refer to: citicoline relieves and/or improves symptoms of respiratory chain dysfunction;
preferably, the symptoms are selected from: levels of PC and/or PE and/or ATP decreased, and/or, levels of NDUFA8 and/or UQCRC2 and/or COXIV decreased.

In specific embodiments, the effective concentration for alleviating and/or improving respiratory chain dysfunction with citicoline is 500 mg/kg.

According to production needs and practical requirements, and based on teachings of the present invention, a person skilled in the art can add various pharmaceutically acceptable excipients/excipients to citicoline, to prepare various dosage forms (including but not limited to oral liquid, injection, tablet, powder, granule, aerosol, spray, etc.) for easy sales or promotion.

Any use of citicoline in preparing drug or product for treating respiratory chain dysfunction, and/or any use of citicoline for treating and/or relieving and/or improving of respiratory chain dysfunction, and/or any action of placing of citicoline in packaging boxes labeled with therapeutic/relief/improvement effects for respiratory chain dysfunction, falls within protection scope of the present invention.

Examples Group 3. shRNA of the Present Invention

This group of examples provides a shRNA causing respiratory chain dysfunction. All examples of this group possess the following common features: its reverse complementary sequence is shown as SEQ ID NO. 2.

In specific examples, DNA sequence corresponding to the shRNA is shown as SEQ ID NO. 1;
preferably, said causing respiratory chain dysfunction refers to levels of NDUFA8 and/or UQCRC2 and/or COXIV protein are downregulated by overexpressing shRNA in animal body to lead respiratory chain dysfunction in animal;
preferably, the respiratory chain dysfunction refers to levels of PC and/or PE and/or ATP in the animal body decreased.

Above shRNAs are all designed and synthesized for the first time according to the present invention, and any act of amplifying, synthetizing, producing, manufacturing, selling, promising for sale, using, importing, exporting, secreting, expanding, enriching, connecting, transforming, cloning, expressing any of above shRNAs, and/or any act of using any of above shRNAs for pharmaceuticals or as pharmaceutical ingredients, and/or, Any use of any of above shRNAs for treating falls within the protection scope of the present invention.

Examples Group 4. Use of shRNA of the Present Invention

This group of examples provides use of shRNA in preparing a respiratory chain dysfunction animal model. All examples of this group possess the following common features: a reverse complementary sequence of shRNA is shown as SEQ ID NO. 2.

In some examples, DNA sequence corresponding to the shRNA is shown as SEQ ID NO. 1;
preferably, shRNA induces respiratory chain dysfunction in animal by downregulating levels of NDUFA8 and/or UQCRC2 and/or COXIV;
preferably, the respiratory chain dysfunction refers to a content of PC and/or PE and/or ATP in animal body decrease;
preferably, the animal is selected from: mouse, rabbit, monkey.

Any behavior of using reverse complementary sequences of shRNA such as SEQ ID NO. 2 to prepare respiratory chain dysfunction models, and/or any behavior of using reverse complementary sequences of shRNA such as SEQ ID NO. 2 to cause respiratory chain dysfunction, and/or any behavior of placing reverse complementary sequences of shRNA such as SEQ ID NO. 2 in packaging boxes labeled with respiratory chain dysfunction, falls within the scope of protection of the present invention.

### Examples Group 5. Method of Preparing Respiratory Chain Dysfunction Animal Model of the Present Invention

This group of examples provides a method for preparing a respiratory chain dysfunction animal model. All examples of this group possess the following common features: comprising the following step: downregulating levels of NDUFA8 and/or UQCRC2 and/or COXIV in animal by shRNA whose reverse complementary sequences is shown as SEQ ID NO. 2;
preferably, the method also comprising the following steps: the respiratory chain dysfunction refers to content of PC and/or PE and/or ATP in animal body decreased;
preferably, connecting DNA sequence corresponding to shRNA which is shown as SEQ ID NO. 1 to expression vector to obtain a recombinant expression vector;
preferably, transforming the recombinant expression vector into the host to obtain a transformant;
preferably, transferring the transformant into the animal;
preferably, the expression vector is selected from pAAV-TNT vector;
preferably, the host is selected from a virus or cell;
preferably, the virus is selected from an adenovirus;
preferably, the cell is selected from DH5a competent cell or 293T cell;
preferably, the animal is selected from: mouse, rabbit, monkey.

### Experimental Example 1. Construction of rAAV Vector Carrying shRNA

### 1. Insertion Fragment Synthesis

Synthesizing shRNA and its reverse complementary sequence separately, and dissolving them with TE.

shRNA-Sense:
(SEQ ID NO. 1)
5′TGCTGTTGACAGTGAGCGAGCTCTTCCGATGCTCACTACCG
TGAAGCCACAGATGGGTAGTGAGCATCGGAAGAGCCTGCCTACTGCCTCG
GACTTCAAGGG3′
shRNA-Antisense:
(SEQ ID NO. 2)
5′ CCCTTGAAGTCCGAGGCAGTAGGCAGGCTCTTCCGATGCTC
ACTACCCATCTGTGGCTTCACGGTAGTGAGCATCGGAAGAGCTCGCTCACT
GTCAACAGCA3′

### 2. Carrying Out the Reaction According to System and Temperature in the Instructions:

Nuclease-Free Water 36 μl

Annealing Buffer for DNA Oligos (5×) 10 μl

DNA oligo A (50 μM) 2 μl

DNA oligo B (50 μM) 2 μl

90° C. for 3 minutes, 37° C. for 1 hour, and 4° C. for storage.

### 3. Enzyme Digestion on Vector

The eukaryotic expression vector pAAV-TNT was digested with BamH I and Not I at 37° ° C. for 2 hours with the following reaction system:

10×K Buffer 1 μl

BSA 1 μl

BamHI 1 μl

Not I 1 μl

PAAV-TNT 2 μl

ddH2O 14 μl

### 4. Gel Recovery of Agarose Gel Electrophoresis

Using 1% agarose electrophoresis gel to electrophoresis products of double enzyme digestion, and then using the agarose gel DNA recovery kit of TaKaRa Agarose Gel DNA Purification Kit Ver manufactured by TaKaRa Corporation. 2.0 to recover the products of the double enzyme digestion. The specific operation steps are as follows:
1) Producing 1×TAE buffer agarose gel, and then performing agarose gel electrophoresis on target DNA;
2) cutting agarose gel containing the target DNA under ultraviolet lamp;
3) Weighing the gel block, calculating its volume, and cutting it into pieces;
4) Adding 3 times volume of gel melting solution DR-I Buffer to the gel block, heating and melting the gel block at 75° C.;
5) Adding ½ volume of DR-II Buffer to the gel melting solution, and mixing evenly. When separating DNA fragments less than 400 bp, adding isopropanol to the solution with a final concentration of 20%.
6) Placing Spin Column in reagent kit on Collection Tube;
7) Transferring the solution from above step 5 to a spin column and centrifuging at 12,000 rpm for 1 minute, discarding the filtrate;
8) Adding 500 μl of Rinse A to the Spin Column, centrifuging at 12,000 rpm for 30 seconds, and discarding the filtrate;
9) Adding 700 μl of Rinse B to the Spin Column, centrifuging at 12,000 rpm for 30 seconds, and discarding the filtrate;
10) Repeating step 9;
11) Placing the Spin Column on the Collection Tube and centrifuging at 12,000 rpm for 1 minute;
12)Placing the Spin Column on a new 1.5 ml centrifuge tube, adding 25 μl of preheated Elution Buffer at 60° C. to center of the Spin Column membrane, and placing it at room temperature for 1 minute;
13) Eluting DNA by centrifugation at 12,000 rpm for 1 minute.

### 5. Vector Connection

1) Linking the recovered pAAV-TNT vector and the synthesized DNA fragment using T4 ligase, and incubating overnight at 16° C. according to the following reaction system:

| | | |
|---|---|---|
| 10× T4 DNA ligase | 2.5 | μl |
| DNA fragment: | 0.3 | pmol |
| Vector fragment | 0.03 | pmol |
| T4 DNA ligase | 1 | μl |
| ddH2O | up to 25 | μl |

2) Adding the entire amount (25 μl) to 100 μl of DH5a competent cells and placing them on ice for 30 minutes;
3) After heating at 42ºC for 45 seconds, placing it in ice for 1 minute;
4) Add 500 μl of antibiotic-free LB medium, shaking at 100 rpm for 60 minutes at 37° C.;
5) Culturing on LB plate medium with Amp+, and selecting white monoclonal colonies for identification.

### 6. Plasmid Extraction at Small Amount

Selecting monoclonal colonies, adding them to 3 ml of LB liquid medium containing Amp+, and shaking at 37ºC for 280 rpm overnight. Extracting the plasmid using the EasyPure Plasmid MiniPrep Kit from Beijing TransGen Biotech Company. The specific steps are as follows:
1) Taking 1.5 ml of bacteria that have been cultured overnight and centrifuging at 10,000 g for 1 minute, trying to absorb supernatant as much as possible;
2) Adding 250 μl of colorless solution RB (containing RNase A) and shaking to suspend the bacterial sediment;
3) Adding 250 μl of blue solution LB, gently inverting and mixing up and down for 4-6 times to fully lyse thalli and form a blue and transparent solution;
4) Adding 350 μl of yellow solution NB, gently mixing for 5-6 times until a compact yellow agglutination clot forms, and placing it at room temperature for 2 minutes;
5) Centrifuging for 5 minutes at 15,000 g, carefully absorbing supernatant and adding it to adsorption column;
6) Centrifuging for 1 minute at 15,000 g, discarding the effluent;
7) Adding 650 μl solution WB, centrifuging at 15,000 g for 1 min, and discarding effluent;
8) Centrifuging for 2 minutes at 15,000 g to completely remove the residual WB;
9) Placing the adsorption column in a new Ep tube, add 20 μl of preheated EB at 70° ° C. to the center of the column, and placing it at room temperature for 1 minute;
10) Centrifuging for 1 minute at 10,000 g to elute DNA, and storing the eluted DNA at −20° C.

### 7. Plasmid Identification

Identifying the constructed plasmid by double enzyme digestion and sequencing, to obtain the eukaryotic expression plasmid pAAV-TNT-shRNA. Its structure is shown in FIG. 1.

### 8. Plasmid Extraction at Large Amount

Preparing a 1 L sterile conical flask, adding 300 ml sterile LB medium, and adding ampicillin solution to a final concentration of 100 μg/ml. Adding 50 μl of the desired plasmid (pXX9; phelper; pAAV-TNT-shRNA) and incubating overnight at 280 rpm and 37° C. Extracting the plasmid according to instructions of the OMEGA E.Z.N.A.® Endo-Free Plasmid Maxi Kit. The specific steps are as follows:
1) Collecting bacteria at room temperature by centrifuging 5000 g for 10 minutes;
2) Discarding culture medium and adding 10 ml of Solution I/RNase A mixture, vortexing to completely resuspend;
3) Adding 10 ml Solution II to resuspension mixture, gently inverting and mixing for 10-15 times, and then placing it at room temperature for 2 minutes;
4) Adding 5 ml Buffer N3 in ice bath and gently inverting several times until a white floccular precipitate is formed;
5) Placing HiBind column in collection tube, adding 5 ml Buffer GPS, leaving it at room temperature for 3-10 minutes, centrifuging at 5,000 g for 5 minutes, discarding the filtrate, and placing the column back in the collection tube;
6) Pouring bacterial lysate into syringe filter, placing it for 2 minutes, inserting and pushing piston, and collecting the filtered lysate;
7) Adding 1/10 volume of ETR to the filtered lysate, inverting it 7-10 times, and placing it in an ice bath for 10 minutes;
8) After 5 minutes in a 42° C. water bath, centrifuging at room temperature for 5 minutes at 5,000 g. Transferring supernatant to a new centrifuge tube, adding 0.5 times the volume of absolute ethanol, mixing well, and placing it at room temperature for 2 minutes;
9) Transferring the mixed solution to the activated HiBind column, centrifuging at room temperature for 5 minutes at 5,000 g, and discarding filtrate;
10) Reinstalling binding column into the collection tube, add 10 ml HB Buffer, centrifuging at room temperature for 5 minutes at 5,000 g, and discarding filtrate;
11) Reinstalling the binding column into the collection tube, adding 15 ml DNA Wash Buffer, centrifuging at room temperature for 5 minutes at 5,000 g, and discarding filtrate;
12) Repeating the previous step;
13) Discarding filtrate, reinstalling the binding column in the collection tube, and centrifuging at 6,000 g for 15 minutes;
14) Removing the binding column and drying it at 65ºC for 10 minutes;
15) Installing the binding column into a new centrifuge tube, adding 1-3 ml of preheated 70° C. Endotoxin free Elution Buffer, and placing it at room temperature for 2 minutes. Then centrifuging it at 6,000 g for 5 minutes to elute DNA.
   9. rAAV-Mediated Virus Packaging

Growing 293T cells (human embryonic kidney epithelial cells) to 90%, at 1-2 hours before calcium and phosphate transfection, replacing each culture dish with 12-15 ml fresh medium (containing serum). Adding calcium chloride (CaCl2) to a 50 ml centrifuge tube, then adding the plasmid to form a Ca-DNA mixture. Mixing thoroughly, slowly adding 2×HEBS BUFFER to the Ca-DNA mixture to form a Ca-DNA-P mixture, and shaking the centrifuge tube while adding 2×HEBS. Mixing thoroughly to form calcium phosphate particles. After 8-12 hours, replacing with 18-20 ml serum-free medium. After 72 hours, aspirating and discarding the medium, washing 3 times with PBS, adding 1 ml Tris+NaCl (pH 8.5) to each culture dish, scraping the cells with a spatula, collecting them in a clean centrifuge tube, and freezing at −80° C.

### 10. Virus Purification

Removing cells frozen at −80° C., thawing and dissolving them at 37° C., freeze-thawing repeatedly for 4 times, centrifuging at 8,000 g for 15 min, and placing supernatant into a clean centrifuge tube, discarding the cell pellet.

Mixing thoroughly ethanol precooled to −20° C. with rAAV at a volume ratio of 3:1. After placing in refrigerator at −20° C. for 2 hours, centrifuging at 13,000 rpm for 15 minutes at 4ºC, and discarding the supernatant. After the ethanol evaporates, adding the corresponding volume of Tris+NaCl (pH 8.5) to dissolve the precipitate. Filtering with a Millipore small filter (0.22 μm).

### 11. Virus Titer Assay

Sample processing: 40 μl of rAAV-TNT-shRNA or rAAV-TNT-control virus solution

Proteinase K (20 mg/ml) 5 μl 55° C., reaction for 1 hr;

Phenol: chloroform: isoamyl alcohol 45 μl

Centrifuge at 4° C. for 5 minutes at 12,000 g to recover aqueous phase;

Chloroform 45 μl

Centrifuge at 4ºC for 5 minutes at 12,000 g to recover aqueous phase.

Real-time PCR:

Primer 1 (10 μm) 0.4 μl

Primer 2 (10 μm) 0.4 μl

SYBR Green I Mix 10 μl

ddH2O 8.2 μl

Template 1 μl

95° C. 30 sec-(95° C. 5 sec-60° C. 5 sec-72° C. 20 sec)×40 cycles-Melting Curve

### Experimental Example 2. Establishment of a of Respiratory Chain Dysfunction Model Mice

### 1. Detection of Expression Levels of Mouse NDUFA8 Protein, UQCRC2 Protein, and COXIV Protein:

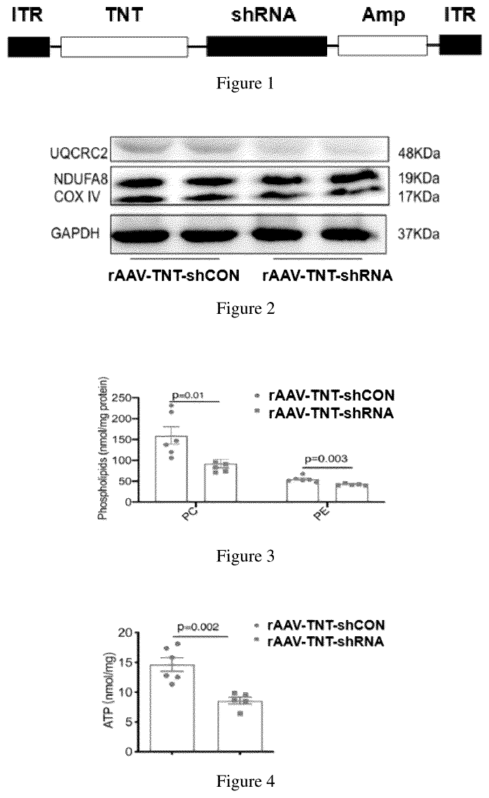

Using 8-week-old C57 mice, injecting rAAV-TNT-shRNA or rAAV-TNT-control through tail vein, with a viral titer of 1×10¹¹ PFU per mouse. Collecting organ tissues until the end of this experiment (12 weeks later). Placing 5 mg of tissue in a centrifuge tube and adding 500 μL of RIPA lysis buffer (pre-added protease inhibitor and phosphatase inhibitor). Homogenizing tissue thoroughly by using a homogenizer. Placing the homogenized tissue on ice for 10 minutes and placing the centrifuge tube in an ultrasonic disrupter for ultrasonic disruption. Then, centrifuging it at 12000 g/min for 20 minutes at 4° C., and aspirating supernatant into another clean centrifuge tube. Measuring protein concentration by using BCA method. Obtaining a standard curve by using the measured standard concentration to calculate protein concentration of sample. Using Western blot to detect expression of NDUFA8 protein, UQCRC2 protein, and COXIV protein, with GAPDH protein as a control. The results are shown in FIG. 2. The expression of NDUFA8 protein, UQCRC2 protein, and COXIV protein in tissues of rAAV-TNT-shRNA mice was significantly lower than that of the control mice rAAV-TNT-control (rAAV-TNT-CON).

### 2. Respiratory Chain Function Detection in Mice:

Analyzing phospholipid metabolism content by targeted ultra-high performance liquid chromatography-mass spectrometry quantitative analysis: weighing about 20 mg of mouse tissue, adding grinding beads to grind thoroughly, adding 70 μL of internal standard (purchased from Avanti Polar Lipids Alabaster, USA), each of which was 100 ng, and dissolving in chloroform/methanol solution (1:1, volume ratio), and 10 μL of BHT solution dissolved in methanol (concentration of 50 mg/mL) to obtain liquid. Then, adding 1 mL of chloroform/methanol solution (1:1, volume ratio) to the liquid at room temperature for 2 minutes to obtain solution. Then, adding 0.45 mL of ultrapure water to the solution, and using a tissue grinder to homogenize. Centrifuging the homogenized liquid at 4ºC for 5 minutes, and collecting the lower layer liquid into a new EP tube. After completing above steps, extracting the upper residue again with chloroform/methanol solution (1:1, volume ratio) and ultrapure water. Combining the two collected lower layer liquids, drying them, and adding 80 μL of chloroform/methanol solution (1:1, volume ratio) to reconstitute them. After filtering with a 0.22 μM filter membrane, entering the detection. The on-machine detection and analysis were completed by Black Technology Co., Ltd. Name was described by Lipid Map (http://www.lipidmaps.org/). ATP content detection: adding 200 μL of lysis buffer per 20 mg of tissue, and then homogenizing the tissue. After homogenization, centrifuging at 12000 g for 5 min at 4ºC, and retaining the supernatant for use. In advance, adding 100 μL of ATP detection working solution to enzyme-labeled tubes, and after allowing the detection working solution to fully consume ATP with placing for 5 minutes, adding 100 μL the collected tissue lysis supernatant samples and standard samples as ATP detection working solution per tube as soon as possible using a pipette gun. Rapidly mixing, determining RLU value by using a chemiluminescence instrument, and drawing a standard curve based on the RLU value and standard samples. Standardizing ATP concentration of each sample based on protein concentration and simultaneously detecting protein concentration.

It's shown by results that, PC and PE (FIG. 3) as well as ATP content (FIG. 4) in the rAAV-TNT-shRNA mice decreased significantly compared to the control mice rAAV-TNT-control (rAAV-TNT-CON).

### Experimental Example 3, the Therapeutic Effect of Citicoline Administration

C57 mice were randomly divided into four groups (rAAV-TnT-shCON+control agent, rAAV-TnT-shRNA+control agent, rAAV-TnT-shCON+CDP-choline, and rAAV-TnT-shRNA+CDP-choline). At 8 weeks of age, injecting the corresponding rAAV virus through tail vein. At 12 weeks of age, feeding mice with 500 mg/kg CDP-choline and control agent (saline) every other day. At the end of the experiment (12 weeks later), collecting heart tissues, and detection indicators were the same as in experimental example 2.

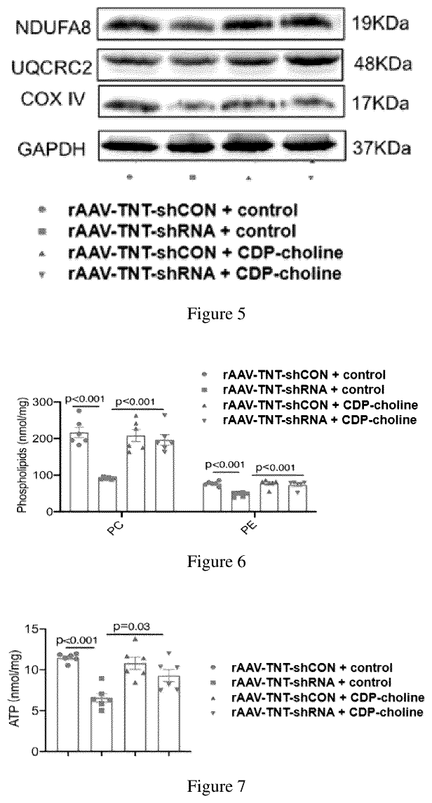

It's shown by results that, compared with the rAAV-TnT-shCON+control group mice, expression levels of NDUFA8 protein, UQCRC2 protein, and COXIV protein (FIG. 5), PC and PE content (FIG. 6), and ATP content in the rAAV-TnT-shRNA+control group mice significantly decreased (FIG. 7). Compared with the rAAV-TnT-shRNA+control group mice, the rAAV-TnT-shRNA+CDP-choline group mice could significantly reverse these changes (FIGS. 5-7), which indicates that treatment with citicoline can improve respiratory chain dysfunction caused by decreased levels of NDUFA8, UQCRC2, and COXIV proteins.

1. Use of citicoline in regulating respiratory chain function related protein level.

2. The use of citicoline in regulating respiratory chain function related protein level according to claim 1, characterized in that, said respiratory chain function related protein is selected from groups consisting of NDUFA8, UQCRC2, COXIV;
said regulating refers to increasing levels of NDUFA8 and/or UQCRC2 and/or COXIV in respiratory chain dysfunction animal model;
and/or, the respiratory chain dysfunction animal model is obtained by overexpressing shRNA in animal body; a reverse complementary sequence of the shRNA is shown as SEQ ID NO. 2;
and/or, DNA sequence corresponding to the shRNA is shown as SEQ ID NO. 1;
and/or, shRNA induces respiratory chain dysfunction in animal by downregulating levels of NDUFA8 and/or UQCRC2 and/or COXIV;
and/or, the respiratory chain dysfunction refers to a decrease in content of PC and/or PE and/or ATP in the animal body;
and/or, the animal is selected from: mouse, rabbit, monkey.

3. Use of citicoline in preparing drug for preventing and treating respiratory chain dysfunction.

4. The use of citicoline in preparing drug for preventing and treating respiratory chain dysfunction according to claim 3, characterized in that, the drug comprises:
pharmacodynamically active ingredients; The pharmacodynamically active ingredients include: citicoline;
and/or, the drug further comprises: pharmaceutically acceptable excipients;
and/or, said preventing and treating refer to: citicoline relieves and/or improves symptoms of respiratory chain dysfunction;
and/or, the symptoms are selected from: levels of PC and/or PE and/or ATP decreased,
and/or, levels of NDUFA8 and/or UQCRC2 and/or COXIV decreased.

5. A shRNA causing respiratory chain dysfunction, characterized in that, its reverse complementary sequence is shown as SEQ ID NO. 2.

6. The shRNA causing respiratory chain dysfunction according to claim 5, characterized in that, DNA sequence corresponding to the shRNA is shown as SEQ ID NO. 1;
and/or, said causing respiratory chain dysfunction refers to levels of NDUFA8 and/or UQCRC2 and/or COXIV protein are downregulated by overexpressing shRNA in animal body to lead respiratory chain dysfunction in animal;
and/or, the respiratory chain dysfunction refers to levels of PC and/or PE and/or ATP in the animal body decreased.

7. Use of shRNA in preparing a respiratory chain dysfunction animal model, characterized in that, a reverse complementary sequence of shRNA is shown as SEQ ID NO. 2.

8. The use of shRNA in preparing a respiratory chain dysfunction animal model according to claim 7, characterized in that, DNA sequence corresponding to the shRNA is shown as SEQ ID NO. 1;
and/or, shRNA induces respiratory chain dysfunction in animal by downregulating levels of NDUFA8 and/or UQCRC2 and/or COXIV;
and/or, the respiratory chain dysfunction refers to a content of PC and/or PE and/or ATP in animal body decrease;
and/or, the animal is selected from: mouse, rabbit, monkey.

9. A method for preparing a respiratory chain dysfunction animal model, characterized in that, comprising the following step: downregulating levels of NDUFA8 and/or UQCRC2 and/or COXIV in animal by shRNA whose reverse complementary sequences is shown as SEQ ID NO. 2.

10. The method for preparing a respiratory chain dysfunction animal model according to claim 9, characterized in that, also comprising the following steps: the respiratory chain dysfunction refers to content of PC and/or PE and/or ATP in animal body decreased;
and/or, connecting DNA sequence corresponding to shRNA which is shown as SEQ ID NO. 1 to expression vector to obtain a recombinant expression vector;
and/or, transforming the recombinant expression vector into the host to obtain a transformant;
and/or, transferring the transformant into the animal;
and/or, the expression vector is selected from pAAV-TNT vector;
and/or, the host is selected from a virus or cell;
and/or, the virus is selected from an adenovirus;
and/or, the cell is selected from DH5a competent cell or 293T cell;
and/or, the animal is selected from: mouse, rabbit, monkey.


SEQUENCE LISTING (2 sequences)

SEQ ID NO: 1 - DNA, 102 bp, synthetic construct
  source 1..102: mol_type = other DNA
tgctgttgacagtgagcgagctcttccgatgctcactaccgtgaagccacagatgggtagtgagcatcggaagagcctgcctactgcctcggacttcaaggg

SEQ ID NO: 2 - DNA, 102 bp, synthetic construct
  source 1..102: mol_type = other DNA
cccttgaagtccgaggcagtaggcaggctcttccgatgctcactacccatctgtggcttcacggtagtgagcatcggaagagctcgctcactgtcaacagca